(19) United States
(12) Patent Application Publication
(10) Pub. No.: US 2009/0259182 A1
(43) Pub. Date: Oct. 15, 2009

(54) WEARABLE LOW PROFILE INFUSION DEVICE

(75) Inventors: Brett CROSS, Seattle, WA (US); Brett J. CARTER, Monroe, WA (US); John M. ADAMS, Kirkland, WA (US); John R. McKENZIE, San Carlos, CA (US); Tom A. SAUL, El Granada, CA (US)

Correspondence Address: GRAYBEAL JACKSON LLP, 155 - 108TH AVENUE NE, SUITE 350, BELLEVUE, WA, 98004-5973, US

(73) Assignee: Calibra Medical, Inc., Redwood City, CA (US)

(21) Appl. No.: 12/469,971

(22) Filed: May 21, 2009

Related U.S. Application Data
(63) Continuation of application No. 11/906,182, filed on Sep. 28, 2007.

(51) Int. Cl. A61M 5/145

(52) U.S. Cl. 604/151

(57) ABSTRACT

A low profile wearable infusion device comprises a generally cylindrical reservoir having a diameter greater than its height and a drive that causes a piston to move an incremental distance within the reservoir to cause the device to dispense a dose of medicament.

### PRIORITY CLAIM

The present application is a Continuation of copending U.S. patent application Ser. No. 11/906,182, filed Sep. 28, 2007, which application is incorporated herein by reference in its entirety.

### BACKGROUND

The present invention relates to infusion devices and more particularly to such devices that enable liquid medicaments to be conveniently and safely self-administered by a patient.

Tight control over the delivery of insulin in both type I diabetes (usually juvenile onset) and type II diabetes (usually late adult onset), has been shown to improve the quality of life as well as the general health of these patients. Insulin delivery has been dominated by subcutaneous injections of both long acting insulin to cover the basal needs of the patient and by short acting insulin to compensate for meals and snacks Recently, the development of electronic, external insulin infusion pumps has allowed the continuous infusion of fast acting insulin for the maintenance of the basal needs as well as the compensatory doses (boluses) for meals and snacks. These infusion systems have shown to improve control of blood glucose levels. However, they suffer the drawbacks of size, cost, and complexity. For example, these pumps are electronically controlled and must be programmed to supply the desired amounts of basal and bolus insulin. This prevents many patients from accepting this technology over the standard subcutaneous injections.

Hence, there is a need in the art for a convenient form of insulin treatment which does not require significant programming or technical skills to implement to service both basal and bolus needs. Preferably, such a treatment would be carried out by an infusion device that is simple to use and mechanically driven negating the need for batteries and the like. It would also be preferable if the infusion device could be directly attached to the body and not require any electronics to program the delivery rates. The insulin is preferably delivered through a small, thin-walled tubing (cannula) through the skin into the subcutaneous tissue similar to technologies in the prior art.

While the idea of such a simple insulin delivery device is compelling, many obstacles must be overcome before such a device may become a practical realty One problem resides in insulin supply. Patients vary greatly on the amount of insulin such a device must carry to provide treatment over a fixed time period of, for example, three days. This is one environment where one size does not fit all. Still further, such devices must be wearable with safety and not subject to possible accidental dosing. Still further, such devices must be capable of delivering an accurately controlled volume of medicament with reliability. While it is preferred that these devices include all of the forgoing features, it would be further preferred if the cost of manufacturing such a device would be economical enough so as to render the device disposable after use. As will be seen subsequently, the devices and methods described herein address these and other issues.

### SUMMARY

The invention provides a wearable infusion device for dispensing fluid such as a liquid medicine like insulin. In some embodiments of the invention, the device comprises a component that causes a piston to move an incremental distance and thereby cause a dose of medicament to be dispensed. The dose dispensed may be equal to a bolus of medicament, or it may be equal to a portion of a bolus. Thus, by moving the piston one or more incremental distances, the device may be used to service the bolus needs of a patient.

The component may be an actuation component that includes an actuation pawl operable to engage a cog in a series of cogs of a drive component. In these embodiments the incremental distance may be the distance between adjacent cogs in the series of cogs, or the distance between two or more cogs in the series of cogs. Thus, the device may be easily used to provide boluses having different amounts of medicament which allows a single device to be easily used by a multitude of people, each requiring a bolus having a different amount of medicament.

In other embodiments of the invention a wearable infusion device comprises a reservoir to hold more than one dose of a medicament; a piston moveable to cause a dose of the medicament to he dispensed; a drive component to cause the piston to move; and an actuation component to limit the distance that the piston moves for each dose dispensed to control the size of the dose.

The drive component may rotate relative to the piston to move the piston. The drive component may also include a thread that engages a thread of the piston and that exerts pressure on the piston's thread when the drive component is rotated relative to the piston.

The actuation component may be operable to rotate the drive component a first incremental distance to cause the device to provide the dose. The actuation component may also include a button that can be moved to cause the drive component to rotate, and a release biased toward a prevent position and movable to a release position, wherein when the release is in the prevent position, the release prevents the button from moving, and when the release is in the release position, the release allows the button to move. In these embodiments, the button and release may be pinched to rotate the drive component.

In still other embodiments, the device may include a lockout component to prevent the piston from moving in a direction that does not cause the medicament to be dispensed, and to lock the piston when the piston reaches its maximum stroke. The lockout component may include a lockout pawl that engages a cog of the drive component to confine movement of the drive component to the direction that causes the device to dispense a dose of fluid, and that engages a slot to lock the drive component's movement when the piston reaches its maximum stoke.

In yet other embodiments of the invention, a wearable infusion device comprises a reservoir for holding fluid to be dispensed, the reservoir being defined by a fixed wall and a side wall extending away from the fixed wall; and a piston disposed in the reservoir and movable relative to the fixed wall to exert pressure on the fluid. The device also comprises a drive component that engages the piston and is operable to move the piston toward the fixed wall, the drive component positioned relative to the piston such that the side wall lies between the drive component and the piston. The device also comprises an output interface in fluid communication with the reservoir.

With the side wall lying between the drive component and the piston, the device can be made small enough to be worn directly on the skin under normal clothing at a location such as the abdomen, without causing discomfort, inconvenience, or creating a hazard, and can thus be used to provide a routine interstitial bolus injection of insulin.

The reservoir may have circular cross-section, and the fixed wall may include an inside surface that is convex relative to the piston.

The piston may be movable toward the fixed wall without rotating relative to the fixed wall. The piston may also include three tabs, each having an end that the drive component engages, and each extending through a respective slot in the side wall to position the respective end for engagement by the drive component.

The invention also provides a system for dispensing a fluid In some embodiments of the invention, the system comprises the device discussed elsewhere herein and a cannula subassembly having a cannula for delivering fluid beneath a patient's skin. The cannula subassembly may be releasably coupled to the output interface of the device and in fluid communication with the reservoir of the device. The output interface may include a needle that is inserted into the cannula subassembly when the device and cannula subassembly are coupled together.

The invention also provides a method for dispensing fluid. In some embodiments of the invention, the method comprises holding the fluid in a reservoir defined by a fixed wall, a side wall extending from the fixed wall and a piston, exerting pressure on the fluid in the reservoir by moving a drive component to move the piston toward the fixed wall, wherein the drive component is positioned relative to the piston such the side wall lies between the drive component and the piston, and allowing fluid in the reservoir to flow through an output interface to reduce the pressure on the fluid in the reservoir.

The activity of exerting pressure on the fluid in the reservoir may include rotating the drive component relative to the piston an incremental distance. The activity may also include moving the piston toward the fixed wall without rotating the piston relative to the fixed wall. The activity may also include rotating the drive component an incremental distance in a first direction and preventing the drive component from moving in a direction opposite the first direction.

In other embodiments of the method, the method may include locking the drive component when the piston reaches its maximum stroke.

### BRIEF DESCRIPTION OF THE DRAWINGS

The invention, together with further features and advantages thereof, may best be understood by making reference to the following description taken in conjunction with the accompanying drawings, in the several figures of which like reference numerals identify identical elements, and wherein:

FIG. 1 shows a top perspective view of an exemplary wearable infusion device. FIG. 2 shows a bottom perspective view of the wearable infusion device of FIG. 1.

FIG. 3 shows an exploded, cross-sectional view of a portion of the wearable infusion device of FIG. 1, along the plane indicated by the line 3-3 in FIG. 1.

FIG. 4 shows a cross-sectional view (not exploded) of the wearable infusion device of FIG. 1, along the plane indicated by the line 3-3 in FIG. 1.

FIG. 5 shows two components (not coupled) of the wearable infusion device, and fluid flow through a portion of the wearable infusion device in FIG. 1.

FIG. 6 shows a top view of an exemplary actuation component included in the wearable infusion device of FIG. 1.

FIG. 7 shows a top view of an exemplary lockout component included in the wearable infusion device of FIG. 1.

FIG. 8 shows a cross-sectional view of an exemplary fill port included in the wearable infusion device of FIG. 1.

### DESCRIPTION

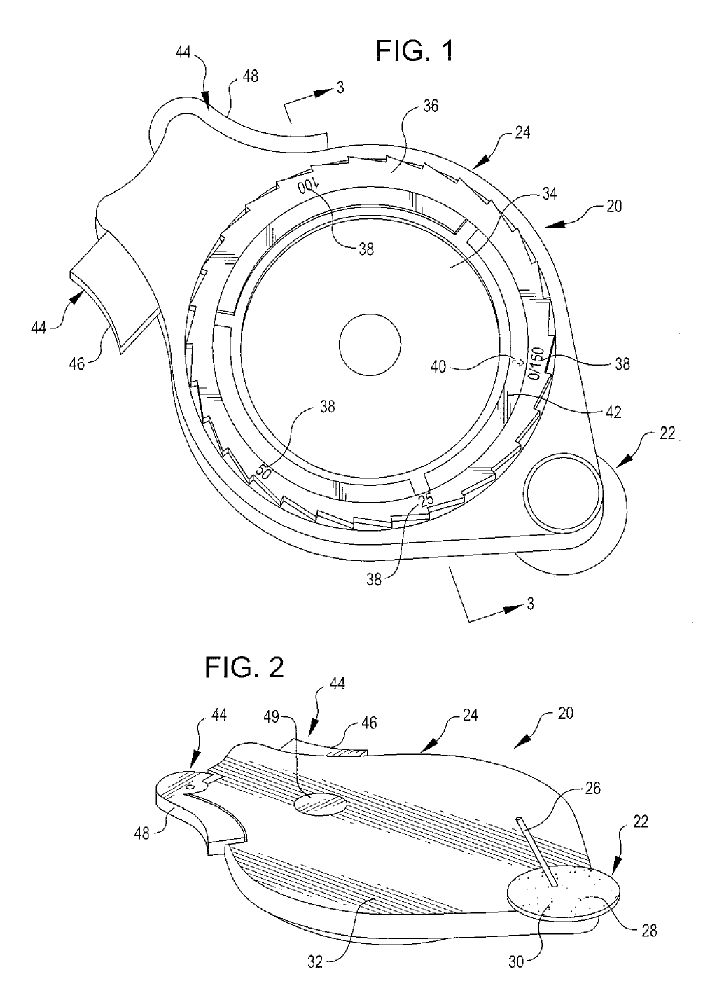

Referring now to FIGS. 1 and 2, they show a wearable infusion device 20 embodying the present invention. The assembly 20 is configured to be worn on a patient's skin and, when operated, provides a patient a bolus injection of any desired fluid, such as insulin for treating diabetes The assembly 20 is small enough to be worn directly on the skin under normal clothing at a location such as the abdomen, without causing discomfort, inconvenience, or creating a hazard.

The wearable infusion device 20 includes a cannula subassembly 22 to deliver the fluid into a patient's body, and a source subassembly 24 to hold the fluid and supply the fluid to the cannula subassembly 22. In some embodiments, the cannula subassembly 22 and the source subassembly 24 are initially separate units that are releasably coupled together to form the wearable infusion device 20. In some of these embodiments, one mounts the cannula subassembly 22 to a patient's body before coupling the cannula subassembly 22 to the source assembly 24. U.S. patent application Ser. No. 11/803,007, filed May 11, 2007 and titled INFUSION ASSEMBLY, which is hereby incorporated by reference for all of its teachings and disclosures, discusses in greater detail the cannula subassembly 22 and mounting the subassembly 22 to a patient. In other embodiments, the cannula subassembly 22 and source subassembly 24 are not separate units that must be mounted to each other to form the wearable infusion device 20.

As may be noted, the cannula subassembly 22 includes a cannula 26 projecting from a first or bottom surface 28 so that when the cannula subassembly 22 is mounted on a patient's skin, the cannula 26 projects to beneath the patient's skin. The surface 28 includes an adhesive coated portion 30 to permit the cannula subassembly 22 to adhere to a patient's skin.

The source subassembly 24 similarly includes an adhesive coated bottom surface 32 that permits the source subassembly 24 to adhere to the patient's skin. It is to be particularly noted that, in accordance with one aspect of the present invention, the adhesive coating 30 of the cannula subassembly 22 is separate and independent from the adhesive coating 32 of the source subassembly 24 Hence, each may be independently adhered to the patient's skin.

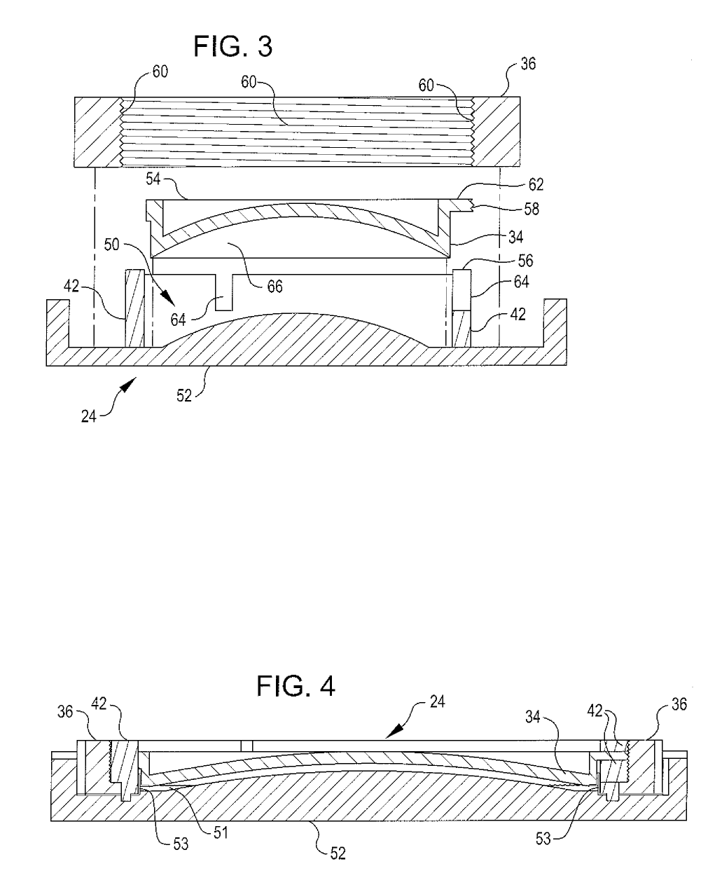

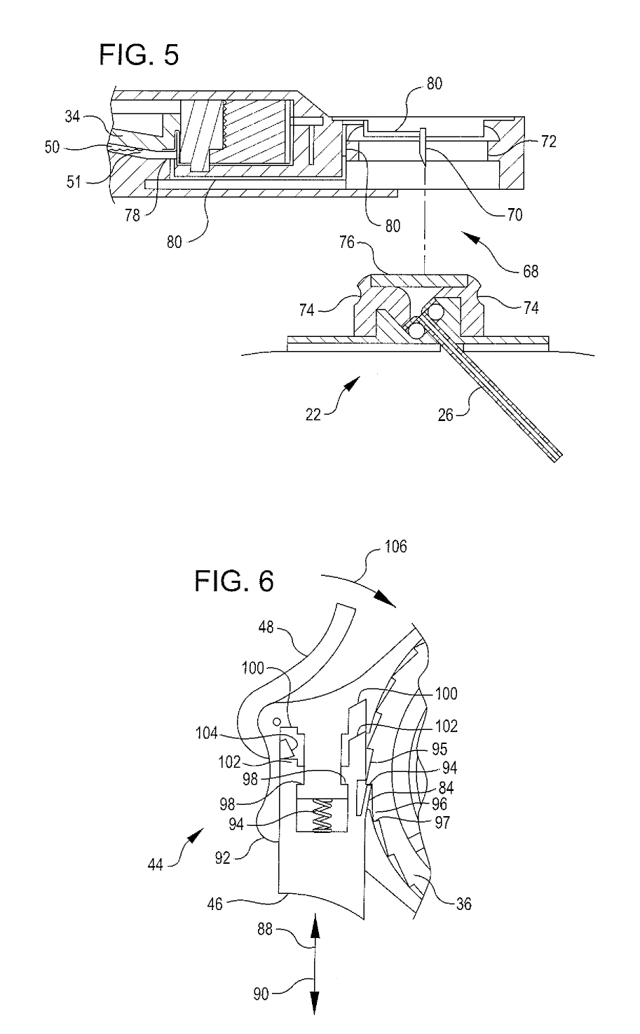

The source subassembly 24 includes a reservoir (not. shown in FIGS. 1 and 2, but shown in FIGS. 3 and 4) to hold fluid, a piston 34 that caps the reservoir, and a drive component 36 to move the piston 34 relative to the reservoir. As discussed in greater detail in conjunction with FIGS. 3 and 4, when the drive component 36 moves the piston 34 toward the fluid in the reservoir (into the paper as shown in FIG. 1 of this embodiment), the piston 34 exerts pressure on the fluid. In response to the pressure, some of the fluid flows through an outlet and conduit (not shown in FIGS. 1 and 2 but shown in and discussed in greater detail in conjunction with FIG. 5) toward the cannula subassembly 22.

The source subassembly 24 can also include a gauge that provides a patient with information relating to the amount. of fluid in the reservoir that is available for future delivery. In this and other embodiments, the drive component 36 includes markings 38 that, in combination with a mark 40 on a wall 42 of the reservoir, show a patient how full the reservoir is at all times, i.e. how many boluses remain available for future use Here, the marking 38 that is aligned with the mark 40, reveals that there are either 150 units (1.5 cc) available for future delivery, or 0 units (0 cc) available depending on whether the wearable infusion device has been used.

The source subassembly 24 also includes an actuation component 44 that moves the drive component 36 an incremental distance. In this and other embodiments the actuation component 44 rotates the drive component 36 clockwise as viewed in FIG. 1. In response, the drive component 36 moves the piston 34 an incremental distance, which may or may not be equal to the incremental distance that the drive component 36 is moved The piston 34 then exerts pressure on the fluid in the reservoir to dispense a dose. In this and other embodiments, the actuation component 44 includes a drive button 46 and a release button 48. As discussed in greater detail in conjunction with FIG. 6, when a bolus of fluid is desired, one first moves the release button 48 to a release position and holds the button at this position. Then, to move the drive component 36 an incremental distance one moves the drive button 46 through its full stroke, i.e. until the button 46 won't move anymore. If the bolus desired is greater than the dose dispensed by moving the drive component 36 a single increment of distance, one can repeatedly move the drive button 46 to move the drive component 36 the required distance. When the release button 48 is not in the release position, the drive button 46 can not be moved to prevent accidental actuation of the device, and thus prevent accidental delivery of a dose

As discussed in greater detail in conjunction with FIG. 8, the source subassembly 24 can also include a port 49 to fill the source subassembly 24 with fluid. This permits the source subassembly 24 to be filled with the desired fluid just before mounting the source subassembly 24 to a patient's skin. The port 49 also permits the source subassembly 24 to be reused, if desired.

FIG. 3 shows an exploded, cross-sectional view of a portion of the wearable infusion device 20 (FIG. 1), along the plane indicated by the line 3-3 in FIG. 1. FIG. 4 shows a cross-sectional view (not exploded) of the wearable infusion device 20 (FIG. 1), along the plane indicated by the line 3-3 in FIG. 1. As can be seen in FIGS. 3 and 4, the source subassembly 24 is configured to provide a low profile so that the wearable infusion device 20 can be easily held directly on the skin and under normal clothing at a desirable location such as the abdomen, without generating attention to the assembly 20 or a hazard, or without causing discomfort or inconvenience.

As previously mentioned, the source subassembly 24 includes the piston 34, the drive component 36, and a reservoir 50 to hold fluid to be dispensed. The reservoir 50 is defined by a fixed wall 52 and a side wall 42. In this and other embodiments, the fixed wall 52 is a bottom wall, and the piston 34 caps the reservoir 50 and is moved toward the bottom wall by the drive component 36 to exert pressure on the fluid 51 (FIG. 4) that is held in the reservoir 50. The bellows 53 (FIG. 4) seals the interface between the piston 34 and the sidewall 42. To keep the profile of the reservoir 50 low, the top surface 54 of the piston 34 remains even with or below the top surface 56 of the side wall 42, and the top surface of the drive component 36 also remains even with or below the top surface 56 of the side wall 42. Thus, the sidewall 42 lies between the piston 34 and the drive component 36.

The drive component 36 can engage the piston 34 in any desired manner to move the piston relative to the fixed wall 52. In this and other embodiments, the piston 34 includes threads 58, and the drive component 36 includes threads 60 that threadingly engage the piston's threads 58. The piston's threads 58 are located at the end of a tab 62 (three shown in FIG. 1 but only one shown in FIGS. 3 and 4) that extends through a respective slot 64 (three shown in FIG. 1 but only two shown in FIG. 3). The slots 64 are configured to confine the piston's movement to two directions—toward or away from the fixed wall 52. Thus, when the drive component rotates around the sidewall 42, the drive component's threads 60 exert pressure on the piston's threads 58 and thereby move the piston 34 toward the fixed wall 52.

The reservoir 50, piston 34 and drive component 36 can be configured as desired to provide any desired dose per incremental distance that the piston 34 is moved. In this and other embodiments, the dose amount dispensed is a function of the incremental distance that the piston 34 moves multiplied by the projected area of the piston's surface 66 onto a plane oriented perpendicular to the direction of the piston's movement. Therefore, to generate a large dose the diameter of the piston's projected area can be increased, the length of the incremental distance can be increased, or both. Similarly, to generate a small dose the diameter of the piston's projected area can be decreased, the length of the incremental distance can be decreased, or both. In this and other embodiments, the length of the incremental distance can be increased or decreased by increasing or decreasing, respectively, the pitch of the threads 58 and 60. In this manner, the source subassembly 24 can be configured to provide a dose that is equivalent to a desired bolus, and thus a patient need only move the drive button 46 once to obtain the desired bolus.

FIG. 5 shows a view of the cannula subassembly 22 and the output interface 68 of the source subassembly 24 separate from each other. FIG. 5 also shows the fluid flow from the reservoir 50 to the output interface 68. As previously mentioned, in this and other embodiments the cannula subassembly 22 is releasably coupled to the source subassembly 24 via the output interface 68.

The output interface 68, in this and other embodiments, includes a needle 70, and an annular ring 72 configured to nest in the detent 74 of the cannula subassembly 22, when the subassembly 24 is inserted into the output interface 68

To releasably couple the cannula subassembly 22 with the output interface 68, the cannula subassembly 22 is first aligned with and inserted into the output interface 68., As the cannula subassembly 22 is inserted into the output interface 68, the needle 70 pierces the septum 76 of the cannula subassembly 22, and the annular ring 72 enters the detent 74 of the cannula subassembly 22. When the cannula subassembly 22 is fully inserted within the output interface 68, the needle 70 has established fluid communication with the cannula 26, and the annular ring 72 nests within the detent 74 to hold the cannula subassembly 22 to the output interface 68. With fluid communication established between the needle 70 and cannula 26, fluid 76 in the reservoir 50 can flow through the outlet 78, through the conduit 80, through the needle 70, and through the cannula 26 to enter a patient's body. To separate the cannula subassembly 22 from the output interface 68, one exerts force on the cannula subassembly 22 until the annular ring 72, the detent 74, or both, sufficiently deform to allow the cannula subassembly 22 to be withdrawn from the output interface 68.

FIG. 6 shows a top view of an exemplary actuation component included in the wearable infusion device of FIG. 1. As previously mentioned, the actuation component 44 moves the drive component 36 an incremental distance, which may or may not be equal to the incremental distance that the piston 34 (FIGS. 1, 3 and 4) moves in the reservoir 50 (FIG. 3).

In this and other embodiments the actuation component 44 includes a drive button 46 having an actuation pawl 84. The drive button 46 is configured to move in two directions 88 and 90 relative to the body 92 of the source subassembly 24. When the drive button 48 moves in the direction 88, the actuation pawl 84 exerts pressure on a contact surface 94 of a cog 95 disposed on the drive component 36. The pressure causes the drive component to rotate (clockwise as shown in FIG. 6), which in turn causes the piston 34 to move in the reservoir 50. When the drive button 46 moves in the direction 90, the actuation pawl 84 slides past an adjacent cog 96 and is positioned to exert pressure on the contact surface 97 of cog 96 when the drive button is again moved in the direction 88. A spring 94 urges the drive button 46 to move in the direction 90, and the shoulders 98 prevent the drive button from moving too far in this direction. An end wall 100 in the body 92 prevents the drive button from moving too far in the direction 88. The full stroke of the drive component 46 is the movement of the drive component 46 from the position shown in FIG. 6 to the position where an end 102 of the drive component 46 contacts the end wall 100.

As can be seen from FIG. 6, the drive button 46 and the drive component 36 are configured to engage each other such that moving the drive button 46 through its full stroke causes the drive component 36 to rotate the distance between the contact surfaces 94 and 97 of adjacent cogs 95 and 96, respectively. Thus, in this embodiment, the incremental distance traveled by the drive component 46 is the distance between contact surfaces of adjacent cogs. In other embodiments, the drive component 36 can include more cogs on the periphery of the drive component 36 to allow a patient more control over the dose provided by a single movement of the drive button 46 For example, if the drive component 36 shown in the figures had twice as many cogs, the incremental distance traveled by the drive component 36 would remain the same but would comprise the distance between the contact surface of every other cog. Therefore, a patient could move the drive button 46 through half of its full stroke to inject a small dose of fluid, or through the drive button's full stroke to inject a larger dose.

The actuation component 44 also includes a release button 48 that must be moved from a prevent position (shown in FIG. 6) to a release position (not shown) before a patient can move the drive button 46 to dispense a dose of fluid. In this and other embodiments, the release button 48 includes an end 104, and is pivotally attached to the body 92. In the prevent position, the end 104 contacts the end 102 of the drive button 46 to prevent the drive button from being moved in the direction 88. To move the release button 48 to the release position, a patient rotates the release button in the direction 106. To urge the release button 48 toward the prevent position, a spring (not shown) is disposed between the release button 48 and the body 92.

In this and other embodiments, the drive button 46 and the release button 48 are arranged relative to each other to allow a patient to pinch the two buttons 46 and 48 to move the drive component 36. Pinching allows a patient to create and quickly release a compressive force to generate a snapping movement of the drive button 46, and thus help insure that the drive button 46 is moved through its full stroke.

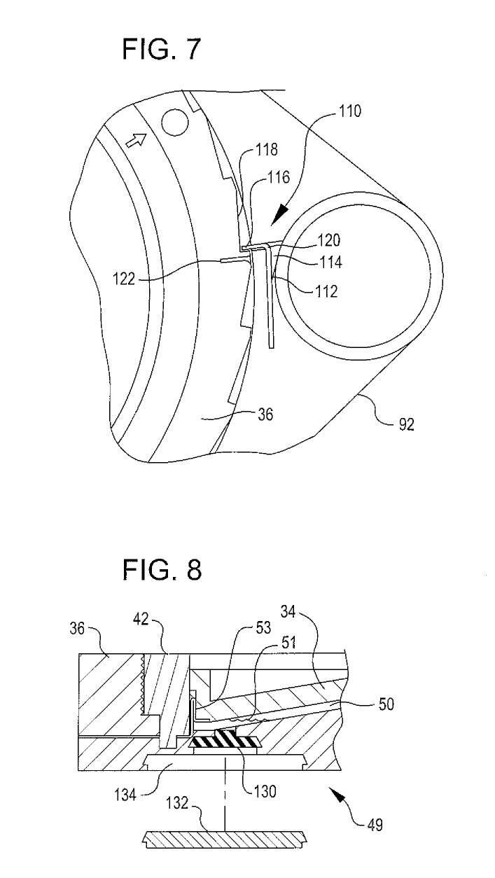

FIG. 7 is a top view of a portion of the wearable infusion device 20 (FIG. 1) that shows an exemplary lockout component 110. The lockout component 110 helps the actuation component 44 restrict the movement of the drive component 36 to a direction (clockwise as shown in FIG. 7) that causes fluid to be dispensed from the source. The lockout component 110 also locks the drive component 36 when the piston 34 reaches the piston's maximum stroke, i.e. the position relative to the fixed wall 52 (FIGS. 3 and 4) of the reservoir 50 (FIG. 3) that the piston 34 does not cross to exert pressure on the fluid. When the piston 34 reaches its maximum stroke, the source subassembly 24 can not dispense another dose and is in effect empty. Thus, the locking of the drive component 36 can represent an empty condition of the source subassembly 24. When locked, the drive component 36 can not move to advance or to withdraw the piston relative to the fixed wall 52.

In this and other embodiments, the lockout component. 110 includes a lockout pawl 112 nested in a receptacle 114 in the body 92 of the source subassembly 24. The lockout pawl 112 includes an end 116 that contacts a cog 118, and the receptacle 114 is configured to allow a portion 120 of the lockout pawl 112 to move relative to the remainder of the lockout pawl 112. As the drive component 36 rotates (clockwise as show in FIG. 7), the end 116 slides relative to the cog 118, and the cog 118 exerts pressure on the end 116. In response to this pressure, the lockout pawl 112 elastically deforms in the receptacle 114 and thereby permits the end 116 to move (to the right as shown in FIG. 7). By elastically deforming, the lockout pawl 112 can remain in contact with the drive component 36 as successive cogs pass the end 116, and can insert the end 116 into the slot 122 when the slot 122 is aligned with the end 116 to lock the drive component 36.

FIG. 8 is a cross-sectional view of a portion of the source subassembly 24 of FIG. 1 that shows an exemplary fill port 49. The fill port 49 permits the source assembly 24 to be filled with the desired fluid just before mounting the source assembly 24 to a patient's skin. The port 49 also permits the source assembly 24 to be reused, if desired.

In this and other embodiments, the fill port 49 includes a septum 130 that a needle can pierce to inject fluid 76 into the reservoir 50 and that can seal the reservoir after the needle is withdrawn. A cover 132 is configured to be snapped into the opening 134 of the fill port 49 to protect the septum 130.

While particular embodiments of the present invention have been shown and described, modifications may be made, and it is therefore intended in the appended claims to cover all such changes and modifications which fall within the true spirit and scope of the invention as defined by those claims.

In the claims:

1-25. (canceled)

26. A low profile wearable infusion device comprising:
a base having a bottom surface including an adhesive adapted to adhere to a patient's skin;
a reservoir for holding a liquid medicament, the reservoir having a generally cylindrical wall upstanding from the base, the cylindrical wall having a height and defining a diameter greater than the height;
a piston moveable within the reservoir in sealing engagement with the cylindrical wall, the piston having at least one extension extending radially outwardly past the reservoir cylindrical wall, the at least one extension terminating in an outer thread;
a ring-shaped drive component circumscribing the reservoir cylindrical wall and having an inner drive thread threadingly engaging the extension outer thread; and
a cannula communicating with the reservoir and extending from the base bottom surface, whereby
as the ring-shaped drive component is rotated an incremental amount substantially less than a complete revolution, the inner thread of the ring-shaped drive component meshes and interacts with the outer thread of the at least one extension of the piston to cause the piston to move an incremental amount within the reservoir to displace an incremental dose of the liquid medicament there from and through the cannula into the patient

27. The device of claim 26, wherein total movement of the piston within the reservoir is defined by substantially one complete revolution of the ring-shaped drive component.

28. The device of claim 26, wherein the reservoir cylindrical wall has at least one slot permitting the at least one extension of the piston to extend radially there through and past the reservoir cylindrical wall.

29. The device of claim 26, wherein the piston includes a plurality of the extensions extending radially outwardly past the reservoir cylindrical wall, each extension terminating in an outer thread to mesh with the inner thread of the ring-shaped drive component.

30. The device of claim 29, wherein the extensions are substantially equally spaced about the piston.

31. The device of claim 30, wherein the reservoir cylindrical wall has a like plurality of slots, each slot permitting a respective given one of the at least one extension of the piston to extend radially there through and past the reservoir cylindrical wall.

32. The device of claim 26, wherein the reservoir has a substantially convex bottom surface and wherein the piston has a substantially concave surface facing and substantially corresponding to the reservoir substantially convex bottom surface.

33. The device of claim 26, wherein the radius defined by the cylindrical wall is at least five times the height of the cylindrical wall.

34. The device of claim 26, further comprising an actuator that is linearly displaceable and arranged, with each actuation, to rotate the ring-shaped drive component the incremental amount.

35. The device of claim 34, wherein the actuator comprises a pawl and wherein the ring-shaped drive component includes a series of cogs arranged to be individually driven by the pawl for rotating the ring-shaped drive component the incremental amount.

36. The device of claim 26, further comprising a lock-out component that limits total rotation of the ring-shaped drive component to substantially one revolution

37. A low profile wearable infusion device comprising:
a base having a bottom surface including an adhesive adapted to adhere to a patient's skin;
a reservoir for holding a liquid medicament, the reservoir having a generally cylindrical wall having a height and defining a diameter greater than the height;
a piston moveable within the reservoir in sealing engagement with the cylindrical wall;
a ring-shaped drive component circumscribing the reservoir cylindrical wall and being operatively associated with the piston; and
a cannula communicating with the reservoir and extending from the base bottom surface, whereby
as the ring-shaped drive component is rotated an incremental amount substantially less than a complete revolution, the piston is caused to move an incremental amount within the reservoir to displace an incremental dose of the liquid medicament there from and through the cannula into the patient.

38. The device of claim 37, wherein total travel of the piston within the reservoir is defined by substantially one complete revolution of the ring-shaped drive component.

39. The device of claim 37, wherein the reservoir cylindrical wall has at least one slot permitting the piston to be operatively associated with the ring-shaped drive component.

40. The device of claim 39, wherein the reservoir cylindrical wall has a plurality of the slots.

41. The device of claim 40, wherein the slots are substantially equally spaced about the cylindrical wall.

42. The device of claim 37, wherein the reservoir has a substantially convex bottom surface and wherein the piston has a substantially concave surface facing and substantially corresponding to the reservoir substantially convex bottom surface.

43. The device of claim 37, wherein the radius defined by the cylindrical wall is at least five times the height of the cylindrical wall.

44. The device of claim 37, further comprising an actuator that is linearly displaceable and arranged, with each actuation, to rotate the ring-shaped drive component the incremental amount.

45 The device of claim 44, wherein the actuator comprises a pawl and wherein the ring-shaped drive component includes a series of cogs arranged to be individually driven by the pawl for rotating the ring-shaped drive component the incremental amount.

46. The device of claim 37, further comprising a lock-out component that limits total rotation of the ring-shaped drive component to substantially one revolution.
